(19) United States
(12) Patent Application Publication
(10) Pub. No.: US 2025/0118409 A1
(43) Pub. Date: Apr. 10, 2025

(54) SYSTEMS AND METHODS FOR PERSONALIZED INSULIN TITRATION

(71) Applicant: Novo Nordisk A/S, Bagsvaerd (DK)

(72) Inventors: Thomas Emil Ryde, Koebenhavn K (DK); Dinesh Krishnamoorthy, Eindhoven (NL); Tinna Bjoerk Aradottir, Copenhagen (DK); Henrik Bengtsson, Taastrup (DK); Dimitri Boiroux, Slangerup (DK)

(21) Appl. No.: 18/730,933

(22) PCT Filed: Jan. 30, 2023
(86) PCT No.: PCT/EP2023/052127

§ 371 Date: Jul. 22, 2024

(30) Foreign Application Priority Data
Jan. 31, 2022 (EP) 22154257.4

(51) Int. Cl. G16H 20/17

(52) U.S. Cl. CPC G16H 20/17

(57) ABSTRACT

A computing system for providing a titration dose guidance a subject to treat diabetes, the system comprises one or more processors and a memory, the memory comprising instructions for executing by the one or more processors: (i) a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and insulin dose size data for the subject, and (ii) a probability algorithm adapted to, based on predicted mean and variance FBG values and an FBG target range, calculate as a function of insulin dose size the probability of hypoglycaemia for the subject.

The present disclosure generally relates to systems and methods for assisting patients and health care practitioners in managing insulin treatment to diabetics. In a specific aspect the present invention relates to systems and methods suitable for use in a diabetes management system providing an optimized personalized basal insulin titration regimen.

### BACKGROUND

Diabetes mellitus (DM) is impaired insulin secretion and variable degrees of peripheral insulin resistance leading to hyperglycemia. Type 2 diabetes mellitus is characterized by progressive disruption of normal physiologic insulin secretion. In healthy individuals, basal insulin secretion by pancreatic B cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours. Years of poorly controlled hyperglycaemia can lead to multiple health complications. Diabetes mellitus is one of the major causes of premature morbidity and mortality throughout the world.

Effective control of blood/plasma glucose can prevent or delay many of these complications but may not reverse them once established. Hence, achieving good glycaemic control in efforts to prevent diabetes complications is the primary goal in the treatment of type 1 and type 2 diabetes. Smart titrators with adjustable step size and physiological parameter estimation and pre-defined fasting blood glucose target values have been developed to administer insulin medicament treatment regimens.

There are numerous non-insulin treatment options for diabetes, however, as the disease progresses, the most robust response will usually be with insulin. In particular, since diabetes is associated with progressive-cell loss many patients, especially those with long-standing disease will eventually need to be transitioned to insulin since the degree of hyperglycemia (e.g., HbA1c≥8.5%) makes it unlikely that another drug will be of sufficient benefit.

The ideal insulin regimen aims to mimic the physiological profile of insulin secretion as closely as possible. There are two major components in the insulin profile: a continuous basal secretion and prandial surge after meals. The basal secretion controls overnight and fasting glucose while the prandial surges control postprandial hyperglycemia.

Based on the time of onset and duration of their actions, injectable formulations can be broadly divided into basal (long-acting analogues [e.g., insulin detemir and insulin glargine] and ultra-long-acting analogues [e.g., insulin degludec (for once-daily administration) and insulin icodec (intended for once-weekly administration]) and intermediate-acting insulin [e.g., isophane insulin] and prandial (rapid-acting analogues [e.g., insulin as part, insulin glulisine and insulin lispro]). Premixed insulin formulations incorporate both basal and prandial insulin components.

Basal insulins will typically be the sole (initial) insulin treatment for type 2 diabetics whereas for type 1 diabetics a basal insulin can be used in combination with a rapid-acting insulin before meals.

Generally, to determine an optimal basal insulin dose for a given patient the patient is titrated starting on an initial safe suggested low dose of basal insulin (typically 10 U/day) which is then increased until Fasting Plasma Glucose (FPG) is within target range, generally 80-130 mg/dl at a dose of typically 40-70 U/day for at type 2 diabetic. Alternatively Fasting Blood Glucose (FBG) values are used. This said, BG and PG are often used interchangeable. Dose adjustments should be more modest and less frequent as the target comes close and down-titration is recommended in case of occurrence of any hypoglycemia.

However, there are significant barriers not only to initiating treatment with insulin but also to optimising the dose and intensifying the regimen, all of which are necessary steps to tailor treatment to individual needs and maintain glycaemic control. The major challenges to this include:

Inter-patient variations: Different types of diabetes, different stages of each type, duration of diabetes, age, different lifestyles and individual physiological differences all contribute to complicate optimization and intensification of treatment regimens.

Intra-patient variations: Furthermore, the overall pathogenesis of diabetes can vary within each patient over time. This is especially true in type 2 diabetes, where the muscle and hepatic cells builds insulin resistance over time, leading to a progressive nature of type 2 diabetes. The complex pathophysiology and progressive nature of T2D means good glycaemic control often becomes increasingly difficult over time, and treatment intensification is often required.

Treatment adherence: The failure to initiate, optimize and intensify basal insulin treatment is also driven by clinical inertia leading to poor treatment adherence. This typically relates to forgetfulness, perceived need for medication, fear of hypoglycaemia, and lack of confidence or uncertainties regarding insulin titration.

Addressing some of the above issues a number of titration concepts and algorithms have been proposed, e.g. as disclosed in US 2019/0147999, US 2020/0227170, US 2019/0272912 and US 2017/213009.

However, there is still a need for a basal insulin titration algorithm that better takes into account differences in the patient population and thus helps patients to reach their treatment goals faster. The algorithm should also provide glycaemic control over time, thus managing diabetes progression over time. Ideally, it should predict the dose needed to reach glycaemic target early in the treatment, thus providing the HCP as well as the patient with insights that enable a meaningful titration, as well as providing an estimate of the risk of hypoglycaemia, hence reducing fear of hypoglycaemia when increasing the dose size gradually to reach target.

### DISCLOSURE OF THE INVENTION

In the disclosure of the present invention, embodiments and aspects will be described which will address one or more of the above objects or which will address objects apparent from the below disclosure as well as from the description of exemplary embodiments. In the context of the present invention, when not used in combination with specific values, the terms FBG and FPG respectively BG and FG are used interchangeable. Correspondingly, in the disclosure of the invention and in the claims the term FBG represents FBG as well as FPG.

In summary, the present invention is based on the realization that a titration algorithm that is personalized to the individual patient will be better suited to achieve the goals of an effective titration process. Thus, a data-driven basal insulin titration algorithm that provides personalized decision support to guide dose adjustments to find the optimal insulin dose is provided.

Correspondingly, in a first aspect of the invention a computing system for providing a titration dose guidance recommendation for a query subject to treat diabetes mellitus is provided, the system comprising one or more processors and a memory. The memory comprises instructions for executing by the one or more processors: (i) a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject, (ii) a probability algorithm adapted to, based on predicted mean and variance FBG values and a pre-defined FBG target range, calculate as a function of insulin dose size the probability of hypoglycaemia respectively hyperglycemia for the subject, and (iii) a policy algorithm adapted to, based on calculated hypoglycaemia respectively hyperglycaemia probabilities, calculate the probability for a corresponding policy target as a function of insulin dose size. The memory further comprises instructions that, when executed by the one or more processors, perform a method responsive to receiving a dose guidance request (DGR), the method comprising the steps of: (a) obtaining for the subject an FBG target to be used as the pre-defined FBG target range for the probability algorithm, (b) obtaining from the subject an update data set comprising a most recent FBG value and a corresponding insulin dose size, (c) using the prediction algorithm: calculating for the subject, based on the update data sat, the mean and the variance of the resulting FBG for the subject as a function of the size of an injected dose of insulin, (d) using the probability algorithm: calculating as a function of insulin dose size the probability of hypoglycaemia respectively hyperglycemia for the subject, (e) using the policy algorithm: calculating the probability for the policy target as a function of insulin dose size, and (f) determining the insulin dose size having the highest calculated probability for meeting the policy target, the determined dose size representing the requested dose size recommendation.

In this way a data-driven insulin titration algorithm is provided that to a high degree assures personalized decision support to guide dose adjustments to find an optimal insulin dose based on a given policy and corresponding target.

The prediction algorithm may be in the form of a probabilistic model with mean μ(x) and variance σ(x) as output, where μ(x) is the mean of the predicted FBG as a function of insulin dose x, and σ(x) is its variance.

The probability algorithm may be in the form of:

$$Prob . of hypo = Pr ( B G \leq {Target}_{low} ) = Φ ( \frac{{Target}_{low} - μ ( x )}{σ ( x )} )$$

$$Prob . of Hyper = Pr ( B G \geq {target}_{high} ) = Φ ( \frac{μ ( x ) - {Target}_{low}}{σ ( x )} )$$

The titration process may be initiated “from scratch” without any patient historic data which would then be accumulated during the process, i.e. the initial update data set may comprise only a most recent FBG value and a corresponding insulin dose size which may be zero. Alternatively, the titration process may be initiated based on a plurality of historic data sets for the subject. For each update a new update data set is created by combining most recent data values with historic data values.

In an exemplary embodiment the computing system comprises a smartphone with a display, the display being controlled to display a determined dose size representing the requested dose size recommendation In a second aspect of the invention a computing system for providing a titration dose guidance recommendation for a query subject to treat diabetes mellitus is provided, the system comprises one or more processors and a memory. The memory comprises instructions for executing by the one or more processors: (i) a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject, and (ii) a policy algorithm adapted to, based on predicted mean and variance FBG values, calculate the probability for a corresponding policy target as a function of insulin dose size. The memory further comprises instructions that, when executed by the one or more processors, perform a method responsive to receiving a dose guidance request (DGR), the method comprising the steps of: (a) obtaining for the subject an FBG target to be used as the policy target for the probability algorithm, (b) obtaining from the subject an update data set comprising a most recent FBG value and a corresponding insulin dose size, (c) using the prediction algorithm: calculating for the subject, based on the update data sat, the mean and the variance of the resulting FBG for the subject as a function of the size of an injected dose of insulin, (d) using the policy algorithm: calculating the probability for the policy target as a function of insulin dose size, and (e) determining the insulin dose size having the highest calculated probability for meeting the policy target, the determined dose size representing the requested dose size recommendation.

In a further aspect of the invention a corresponding method for providing a titration dose guidance recommendation for a query subject to treat diabetes mellitus is provided. The method comprises the steps of (i) obtaining for the subject an FBG target to be used as a pre-defined FBG target range for a probability algorithm, and (ii) obtaining from the subject an update data set comprising a most recent FBG value and a corresponding insulin dose size. The method 35 comprises the further steps of (iii) using a for the subject representative adaptive prediction algorithm: calculating for the subject, based on the update data set, the mean and the variance of the resulting FBG for the subject as a function of the size of an injected dose of insulin, (iv) using a probability algorithm: calculating as a function of insulin dose size the probability of hypoglycaemia respectively hyperglycemia for the subject based on the predicted mean and variance FBG values and the pre-defined FBG target range, (v) using a policy algorithm: calculating, based on the calculated hypoglycaemia respectively hyperglycaemia probabilities, the probability for a corresponding policy target as a function of insulin dose size, and (vi) determining the insulin dose size having the highest calculated probability for meeting the policy target, the determined dose size representing a dose size recommendation.

In a yet further aspect of the invention a computing system for providing a titration dose guidance recommendation for a query subject to treat diabetes mellitus is provided. The system comprises one or more processors and a memory. The memory comprises instructions for executing by the one or more processors: (i) a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject, and (ii) a probability algorithm adapted to, based on predicted mean and variance FBG values and a hypoglycaemia target value, calculate as a function of insulin dose size the probability of hypoglycaemia for the subject. The memory further comprises instructions that, when executed by the one or more processors, perform a method responsive to receiving a dose guidance request (DGR), the method comprising the steps of: (a) obtaining from the subject an update data set comprising a most recent FBG value and a suggested insulin dose size, (b) obtaining for the subject a threshold acceptable probability for hypoglycaemia, (c) using the prediction algorithm: calculating for the subject, based on the update data set, the mean and the variance of the resulting FBG for the subject as a function of the size of an injected dose of insulin, (d) using the probability algorithm: calculating for the suggested insulin dose size the probability of hypoglycaemia for the subject, (e) determining whether the suggested insulin dose size is above or below the threshold acceptable probability, and (f) communicating to the subject whether the suggested insulin dose size is acceptable or should be lowered.

In this way a data-driven insulin algorithm is provided that that can be used to verify whether a given dose suggested by any titration algorithm or an HCP is safe to a desired level of confidence.

### BRIEF DESCRIPTION OF THE DRAWINGS

In the following embodiments of the invention will be described with reference to the drawings, wherein

FIG. 1 illustrates graphically the basic concept of Gaussian process regression,

FIG. 2 illustrates schematically how the probability of hypo- and hyperglycaemia as a function of insulin dose can be determined,

FIG. 3 illustrates the different components of a titration concept based on a Bayesian modelling framework and a policy function,

FIG. 4 shows in silico results for once daily insulin using the proposed Bayesian approach for a first policy function,

FIG. 5 shows in silico results for once daily insulin using the proposed Bayesian approach for a second policy function,

FIG. 6 shows in silico results for once daily insulin using the proposed Bayesian approach for a third policy function,

FIG. 7 shows in silico results for once daily insulin using the proposed Bayesian approach for a fourth policy function,

FIG. 8 illustrates the different components of a Bayesian modelling framework adapted to verify whether a given dose suggested by a given titration algorithm or an HCP is safe, and

FIG. 9 shows a titration look-up table.

### DESCRIPTION OF EXEMPLARY EMBODIMENTS

Overall a diabetes dose guidance system is provided that helps people with diabetes by generating recommended insulin doses. In such a system a given algorithm is used to generate recommended insulin doses and treatment advice for diabetes patients based on BG and insulin dosing history.

Essentially such a system comprises a back-end engine (“the engine”) which is the main aspect of the present invention used in combination with an interacting system in the form of a client and an operating system.

The client from the engine's perspective is the software component that requests dose guidance. The client gathers the necessary data (e.g. CGM data, insulin dose data, patient parameters) and requests dose guidance from the engine. The client then receives the response from the engine.

On a small local scale the engine may run directly as an app on a given user's smartphone and thus be a self-contained application comprising both the client and the engine. Alternatively, the system setup may be designed to be implemented as a back-end engine adapted to be used as part of a cloud-based large-scale diabetes management system. Such a cloud-based system would allow the engine to always be up to date (in contrast to app-based systems running entirely on e.g. the patient's smartphone), would allow advanced methods such as machine learning and artificial intelligence to be implemented, and would allow data to be used in combination with other services in a greater “digital health” set-up. Such a cloud-based system ideally would handle a large amount of patient requests for dose recommendations.

Although a “complete” engine may be designed to be responsible for all computing aspects, it may be desirable to divide the engine into a local and a cloud version to allow the patient-near day-to-day part of the dose guidance system to run independently of any reliance upon cloud computing. For example, when the user via the client app makes a request for dose guidance the request is transmitted to the cloud engine which will return a dose recommendation. In case cloud access is not available the client app would run a dose-recommendation calculation using the current local algorithm. Dependent upon the user's app-settings the user may or may not be informed.

In exemplary embodiments of the invention a Bayesian optimization approach is implemented in which a sequential design strategy for global optimization of black-box functions that does not assume any functional forms is used.

The BG data for a given patient may be provided as e.g. self-measured blood glucose measurements (SMBG) measured at fasting conditions (FBG) or as BG data from a continuous glucose monitor (CGM). Using BG and insulin dosage data allows a data-driven probabilistic model capturing the effect of insulin on blood glucose to be developed.

A Bayesian modelling framework is used that predicts the blood glucose as a function of the insulin, as well as gives a confidence of the predicted function. The confidence of the predicted function is an important variable that can then be used to predict,
1. probability of a hypoglycaemia (BG<=4 mmol/L)
2. probability of reaching target (4<=BG<=7 mmol/L)
3. probability of hyperglycaemia. (BG>=7 mmol/L)

More precisely, a suitable black-box surrogate function such as a Gaussian Process model or a random forest model is used to model the mean and the variance. That is, for any given insulin dose, the model would predict the mean and the variance of the fasting blood glucose.

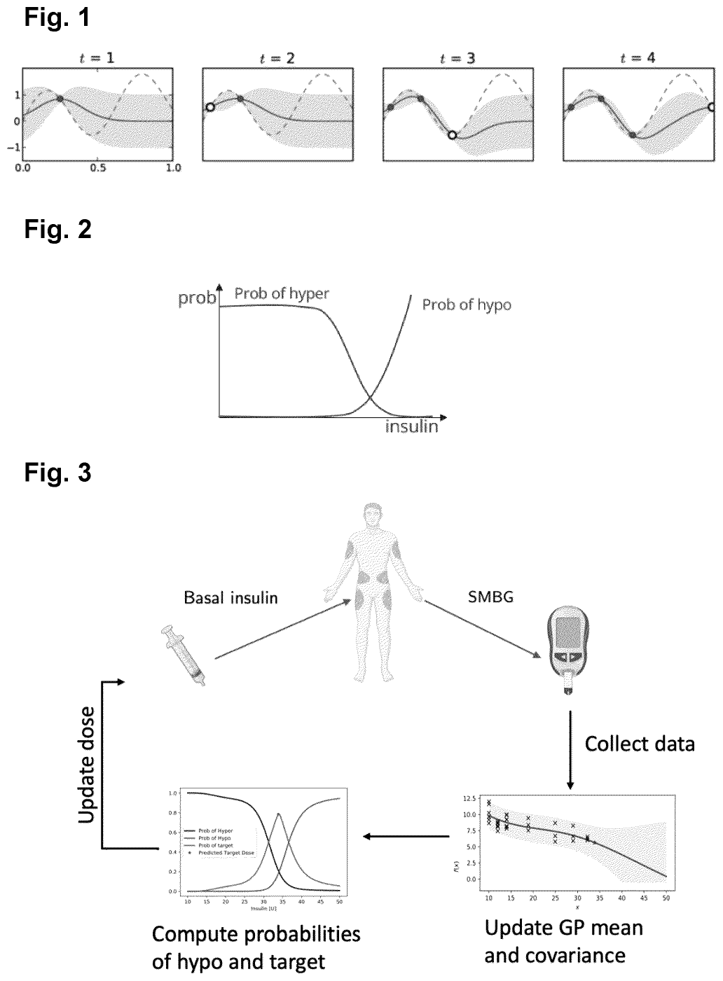

The model is updated for each patient using insulin dose and the fasting blood glucose measurement. Every time a new data point is available (i.e. the dose from the previous period (e.g. last week or day) and the resulting FBG after the dose was given), the mean and the variance of the black-box model is updated by conditioning on the data. The basic idea of Gaussian process regression is graphically illustrated in FIG. 1.

More specifically, the true unknown function is shown in dashed line. Solid dots represent old data points, and the black circle denotes a new data point. A Gaussian Process (GP) model is used as a surrogate to model the true unknown function. The mean of the GP model is shown in full line and the variance is shown in the outlined region. At every time step t, when a new data point is available, the mean and the variance of the GP model are updated by conditioning on the data points.

First and foremost, the use of a Bayesian modelling framework is based on the patient data, which enables the decision-support tool to be personalized for each individual patient. Since the model is conditioned on the patient data, the predicted mean and variance are personalized to each patient.

Secondly, by predicting the probability of hypoglycaemia as a function of insulin dose for a particular patient, this can be used to verify whether the suggested dose is safe. For example, the dose may be suggested by a HCP, a standard-of-care titration algorithm, or any other dose guidance algorithm. If the predicted probability of hypoglycaemia at the suggested insulin dose is lesser than some acceptable value, then the suggested dose can be administered. Predicting the risk of hypoglycaemia would also reduce fear of hypoglycaemia and improve patient confidence in increasing the insulin dose to reach target. FIG. 2 illustrates schematically how the probability of hypo- and hyperglycemia as a function of insulin dose can be determined.

Thirdly, the updated surrogate model (such as the Gaussian process model) can be used to directly titrate the insulin dose. This can be done by constructing a “policy” (also known as acquisition function in the computer science domain) that would suggest what the next insulin dose should be. The decision-making policy will be based on the computed probability of hypoglycaemia and the probability of hyperglycemia. For example, one decision-making policy could be based on the probability of reaching the target zone. This would provide the predicted target dose, i.e. the insulin dose at which the patient is expected to reach the glycaemic target. The predicted target dose could then be used to directly titrate the insulin dose which could help patients reach their targets faster.

Predicting the probability of hypo and probability of target as a function of insulin dose would also provide the patient and the HCP with useful insights.

### Exemplary Embodiments

The above outlined approach can be used for Basal insulin titration for type 1 diabetes or Type 2 diabetes.

It should be noted that dose calculation, i.e., the suggested dose, is made using probabilistic models with mean μ(x) and variance σ(x). μ(x) is the mean of the predicted FPG as a function of insulin dose x, and σ(x) is its variance.

New data sets are used to update the model that forms the basis for dose calculation, however, the calculation per se is based on the probabilistic model. Hence the meaningfulness of the calculation does not depend on how much data is available.

A probabilistic model can be anything that models a phenomenon as well as its uncertainty distribution. That is, at any query point, the probabilistic model returns the value of the function and its confidence bound.

The algorithms have a default one-size-fits-all model that can be termed the “priori mean function”. For a given insulin analogue, some information will be known about what the effect of the insulin on blood glucose is. Typically, the more insulin that is injected, the lower the resulting blood glucose will be. The default one-size-fits-all model will be based on this design. One example is a linear function:

$$B G = m x + c$$

If no data is available, decisions will be calculated based on this default model. Off course this will not be optimal, since this model is not personalized to the individual, and hence will be overly conservative, however, as more data is gathered the default model can be updated and thus adapted to the given person. Alternatively more advanced models may be used just as a given model may have been optimized for a given demographic.

The data set consists of (insulin dose size, FPG) pairs. More precisely, an already injected dose, and its corresponding or resulting FPG. That is, a patient injects insulin on Day 1, and measures the corresponding FBG on Day 2 morning. In this way it can be observed what effect the insulin had on the patient. This observation is what is needed to update the probabilistic model.

So we start with an initial data set D={(0, FPG0)} on Day 0, where for no previously administered insulin, FPG0 is the patients starting blood glucose value, i.e. FPG with no treatment. As there was no data set before the first data set it can also be termed an update data set.

On Day 0: The patient takes initial dose I₁ (e.g. I₁=10U).

On Day 1: patient measures FPG. The updated data set now has D={(0, FPG0), (I₁, FPG1)}

The probabilistic model is updated using D={(0, FPG0), (I₁, FPG1)}, that is, using Bayes rule from classic elementary statistics.

The algorithm then calculates new insulin dose I₂ using the model. Policy 1, 2, 3 or 4 (see below) can be used for this. These policies are based on the mean and the variance of the updated model. Patient takes insulin I₂

On Day 2: patient measures FPG.

The update data set now has D={(0, FPG0), (I₁, FPG1), (I₂, FPG2)}.

The probabilistic model is updated using D={(0, FPG0), (I₁, FPG1), (I₂, FPG2)}.

Then the algorithm calculates new insulin dose I₃ using the model. The patient takes insulin I₃.

On Day 3 and forwards the above procedure is repeated.

On Day N: D={(0, FPG0), (I₁, FPG1), (I₂, FPG2) . . . , (I_{N}, FPGN)}.

In the case of once weekly insulin, Week 1, Week 2, . . . is used instead of Day 1, Day 2 . . . .

As appears, the data set is a table with two columns, insulin dose size, and measured FPG. Every day (or week) a new row is added thereby updating the data set. The fasting blood glucose can be measured using any glucose sensing device, either interstitial, blood, or urine. The mean and the variance of the fasting blood glucose is modelled as a function of insulin dose x.

BG(x)˜GP(μ(x),σ(x))

where x is the insulin dose [U], and BG is the blood glucose in [mmol/L], μ(x) is the mean blood glucose, and σ(x) is the variance.

Using the updated mean and the variance the probabilities of being within the target and the probability of hypoglycaemia as a function of insulin dose can be computed by using the cumulative distribution function, denoted by Φ(·).

$$Prob . of hypo = Pr ( B G \leq {Target}_{low} ) = Φ ( \frac{{Target}_{low} - μ ( x )}{σ ( x )} )$$

$$Prob . of Hyper = Pr ( B G \geq {target}_{high} ) = Φ ( \frac{μ ( x ) - {Target}_{low}}{σ ( x )} )$$

$$For {Target}_{low} = 4 and {target}_{high} = 7 :$$

$$Prob . of hypo = Pr ( B G \leq 4 ) = Φ ( \frac{4 - μ ( x )}{σ ( x )} )$$

$$Prob . of Hyper = Pr ( B G \geq 7 ) = Φ ( \frac{μ ( x ) - 7}{σ ( x )} )$$

As a first example the Kanderain model modified for T2D (Aradottir et al. 2019) is used as a virtual patient. It is to be noted that no model information is used by the algorithm. In the present example basal insulin titration is shown. The target dose can be updated daily, once every n days, or every week. The patient can also be titrated up to the suggested target dose in smaller steps.

To directly titrate the insulin dose a policy function that would guide in updating the insulin dose would have to be constructed. Any policy function constructed using the probability of hypoglycaemia and probability of hyperglycemia can be used.

FIG. 3 illustrates the different components of the above-described concept.

In the following 3 examples of decision-making policies that could be used to suggest the next dose will be described.

$${Policy}_{1} ( x ) = Pr ( 4 \leq B G \leq 7 ) = 1 - Φ ( \frac{4 - μ ( x )}{σ ( x )} ) - Φ ( \frac{μ ( x ) - 7}{σ ( x )} )$$

The first policy is based on computing the probability of being within a pre-defined target range of 4 and 7 mmol/L, i.e. the target is a range. As appears, the first policy does not optimize the probability that a specific target value is reached, e.g. the mean of the range, in contrast it merely optimizes the probability that the achieved BG is within the pre-set range, e.g. it could be 4.1 or 6.9 mmol/L and thus be within target range. The same considerations apply to the second and third policy.

$${Policy}_{2} ( x ) = Prob . of hypo * Prob . of Hyper = Φ ( \frac{4 - μ ( x )}{σ ( x )} ) Φ ( \frac{μ ( x ) - 7}{σ ( x )} )$$

The second policy is based on aggregating the probabilities of constraint feasibility (in this case the low and high limits on BG values).

$${Policy}_{3} ( x ) = min ( ( 1 - Φ ( \frac{4 - μ ( x )}{σ ( x )} ) ) , ( 1 - Φ ( \frac{μ ( x ) - 7}{σ ( x )} ) )$$

The third policy is based on trading-off between the hypoglycaemia and hyperglycaemia probability, i.e. choosing the point where the probability of hypoglycaemia and hyperglycaemia intersects.

Potentially further policy functions could be constructed based on the probability of hypoglycaemia and hyperglycemia. Any such meaningful policy could be used.

Additionally, policy functions could also be based on the mean of the gaussian process model. For example,

$${Policy}_{4} ( x ) = - {( μ ( x ) - target )}^{2}$$

where μ(x) is the mean of the Gaussian process model. As appears, for the fourth policy the target is a single value and not a range.

To incorporate the information of the probability of hypoglycaemia and hyperglycemia in policy 4 it could also be scaled with the probability of feasibility, i.e. by multiplying policy 4 with policy 2.

Once a policy function is chosen, the suggested dose is given by the insulin value that maximizes the policy function.

$$Suggested dose = x *= arg {max}_{x} Policy ( x )$$

In the following implementation of the different policies will be illustrated using a series of plots. In all the plots, lower subplot (a) shows the Bayesian model that is updated earlier in the titration procedure with only a few past data points. As shown in the upper subplot (a), using the Gaussian process model, the probabilities of hypoglycaemia and hyperglycemia as a function of insulin dose are also computed (every 3 days in this case). As the titration process continues more data points are obtained and the gaussian process model is updated with each new data point. The lower subplot (b) shows the updated GP model with more data points after some weeks, and the corresponding probability of hypoglycaemia and hyperglycaemia is shown in the upper subplot (b). Subplot (c) shows the SMBG measurements, and the insulin dose administered on each day using the proposed Bayesian titration approach. The Bayesian titration approach was initiated on Day 20, and it can be seen that this helps personalize the titration to the specific patient and thus reach the glycaemic target quickly.

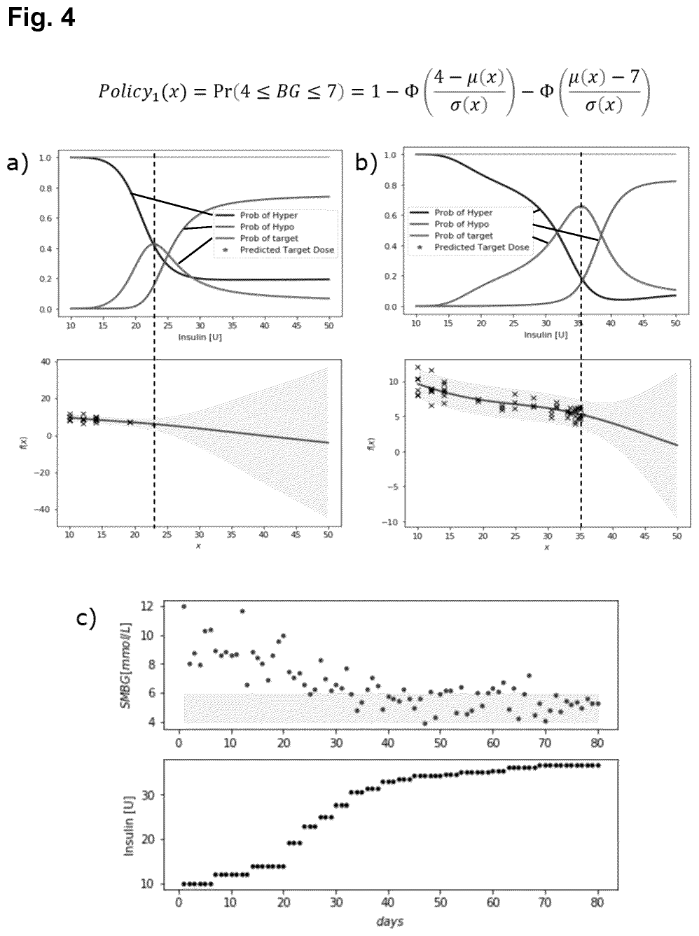

FIG. 4 shows for policy 1 in silico results for once daily insulin using the proposed Bayesian approach at two different days, one earlier in the titration procedure (a), and the other later in the titration procedure (b). As more data points become available the Gaussian process captures the insulin-glucose effect more accurately, and hence suggest a more appropriate target dose, this as shown in (c).

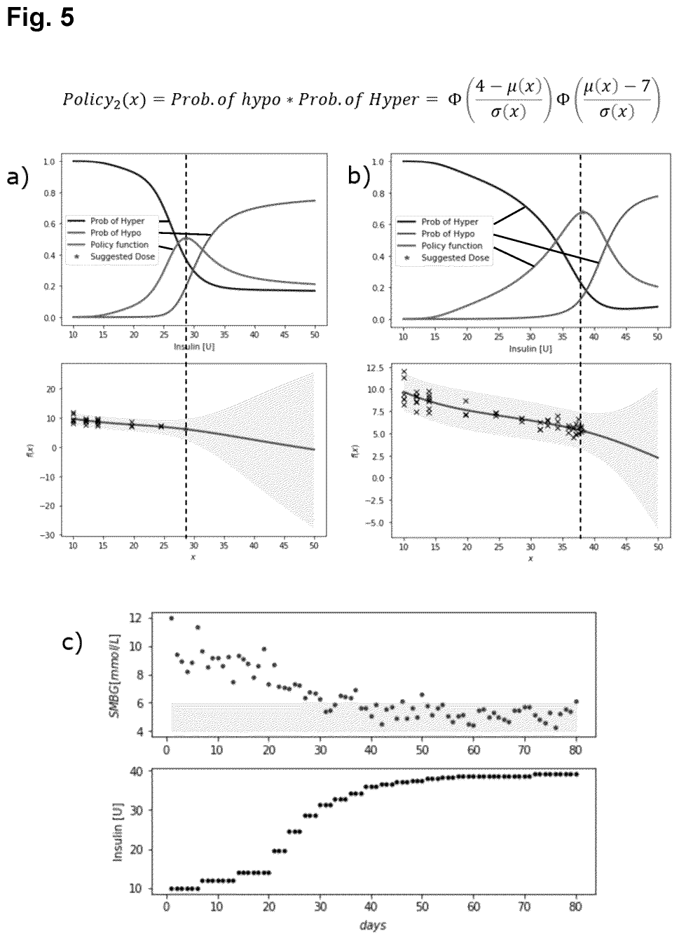

FIG. 5 shows for policy 2 corresponding in silico results for once daily insulin using the proposed Bayesian approach at two different days.

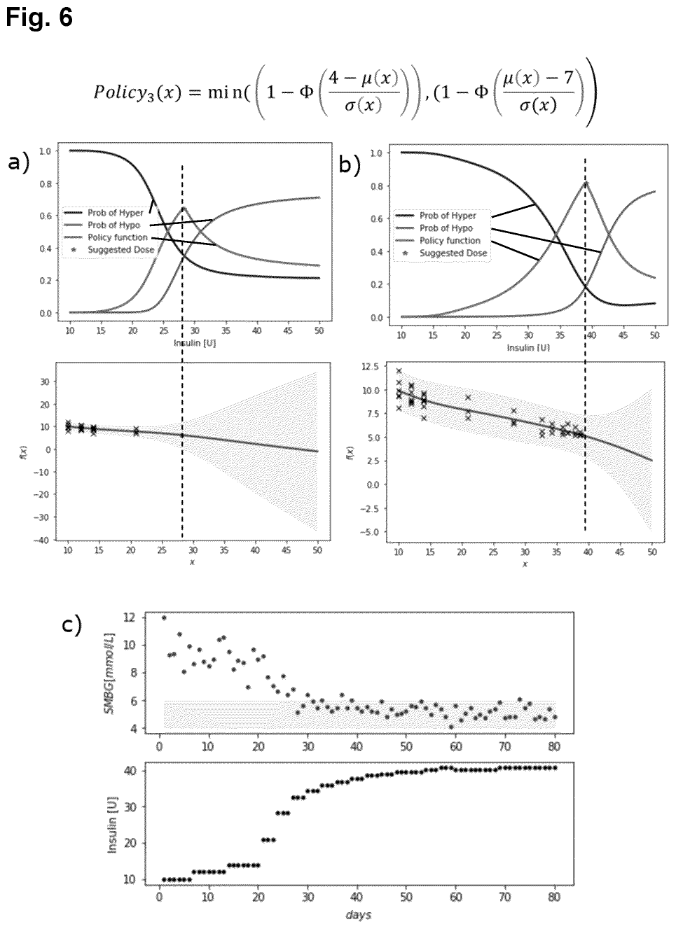

FIG. 6 shows for policy 3 corresponding in silico results for once daily insulin using the proposed Bayesian approach at two different days.

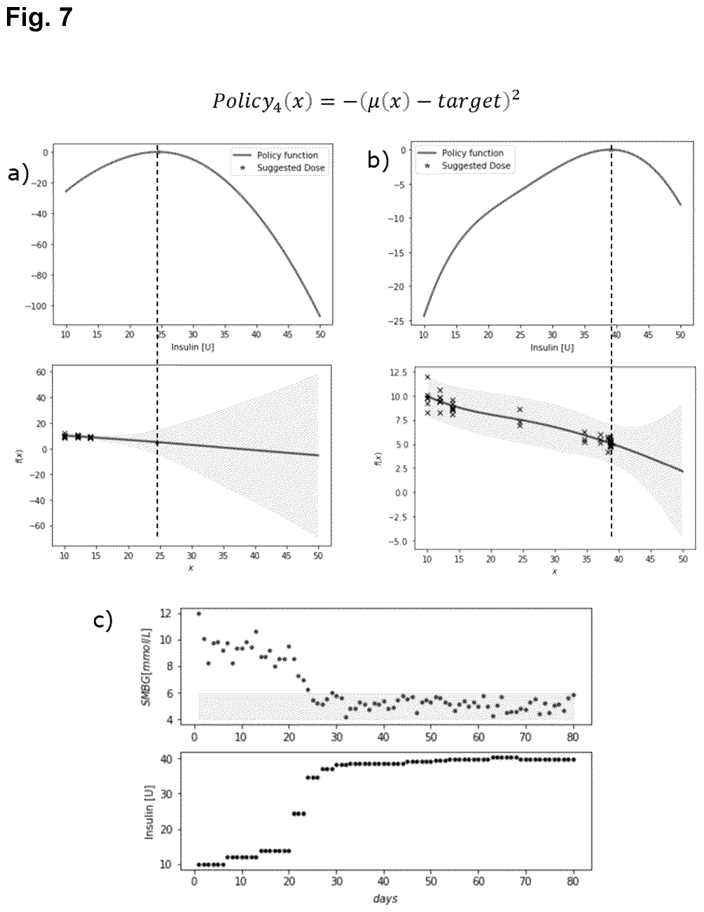

FIG. 7 shows for policy 4 corresponding in silico results for once daily insulin using the proposed Bayesian approach at two different days.

The above given examples show that the estimation of probability of hypoglycaemia gives the HCP and the patient a meaningful insight into the risk of hypoglycaemia for the suggested dose. These can be used to construct a policy that can be used to guide in the insulin titration.

This approach requires choosing an initial prior mean function to capture the effect of insulin on the blood glucose. This can be chosen suitably using domain knowledge. For example, for a given insulin analogue, we will have some information about what the effect of the insulin on blood glucose is. Typically, the more insulin, the lower is the blood glucose. Therefore we can heuristically choose a linear function

$$B G = m x + c$$

Where m is the slope, and c is the intercept.

The chosen model can be a one-size-fits-all model that captures the effect of insulin on blood glucose. This initial one-size-fits-all prior model can then be personalized to the patient every time a new data point is available.

As more data points are available, the slope and the intercept of the prior must also be ideally updated such that the prior can be extrapolated in a more meaningful way where data points are not available.

This approach can be used with once daily insulin analogues, or once weekly insulin analogues.

The below table 1 shows an example of numerical values using the Bayesian approach in combination with the above-described policy 3 (with no noise):

| Index | SMBG (fasting) | Suggested | Predicted prob. of |
|---|---|---|---|
| (day) | [mmol/L] | dose [U] | Hypo at suggested dose |
| 0 | 8.58 | 19.29 | 0.22 |
| 1 | 7.66 | 25.35 | 0.28 |
| 2 | 6.74 | 30.20 | 0.26 |
| 3 | 6.11 | 33.03 | 0.23 |
| 4 | 5.77 | 34.67 | 0.21 |
| 5 | 5.63 | 35.45 | 0.18 |
| 6 | 5.53 | 36.26 | 0.19 |
| 7 | 5.44 | 36.67 | 0.17 |
| 8 | 5.44 | 37.47 | 0.16 |
| 9 | 5.42 | 38.28 | 0.15 |

In the shown model example the actual dose taken by the patient corresponds exactly to the dose suggested by the algorithm, e.g. 25.35 U. In a real-world scenario the doses would be rounded to integer-sized doses. The points are ‘split in time’ so the suggested dose in row x is matched with the SMBG in row x+1.

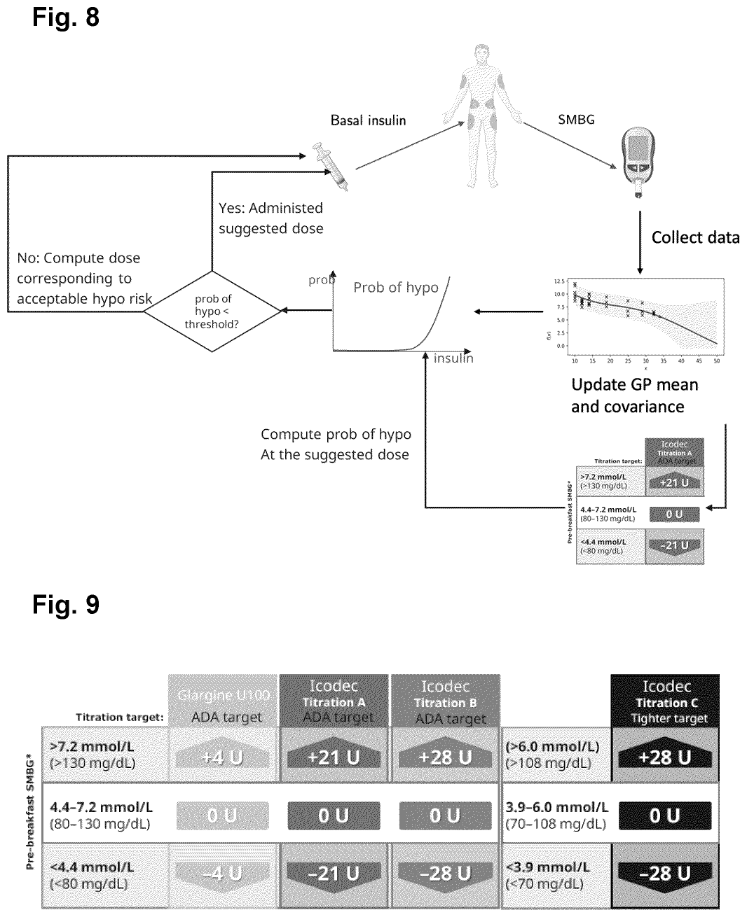

In a second exemplary embodiment the Bayesian modelling framework is used to verify whether a given dose suggested by any titration algorithm or an HCP is safe, this as illustrated in FIG. 8. With each new data point, the gaussian process model can be updated to develop a personalized model that predicts the mean and variance of the blood glucose as a function of the insulin dose. This probabilistic model allows the probability of hypoglycaemia to be predicted as explained above. The predicted probability of hypoglycaemia can be used to verify whether a suggested dose increase is safe. The dose update may either come from an HCP or any given titration algorithm.

The above approach was tested using insulin icodec (a long-acting basal insulin analogue intended for once-weekly treatment being developed by Novo Nordisk A/S for the treatment of type 1 and type 2 diabetes) clinical trial data from clinical trial NN4465, where the titration was done according to a look-up table. An example of such a look up table is shown in FIG. 9.

In the below table 2 a numerical example using a simple titration algorithm in combination with the Bayesian approach is shown. Note that the example starts from week 14 as the uncertainty measure is more reliable after some weeks.

| Index | SMBG | Prev. insulin | New insulin | Predicted prob. of |
|---|---|---|---|---|
| (week) | (fasting) | dose [U] | dose [U] | Hypo at new dose (%) |
| | 7 | | | |
| | 5.6 | | | |
| 14 | 6.5 | 420 | 434 | 3.40 |
| | 5.4 | | | |
| | 6.6 | | | |
| 15 | 6.6 | 434 | 448 | 4.49 |
| | 6.9 | | | |
| | 6.3 | | | |
| 16 | 6.6 | 448 | 462 | 5.39 |
| | 5.9 | | | |
| | 6.7 | | | |
| 17 | 6.2 | 462 | 476 | 7.15 |
| | 6.3 | | | |
| | 7.3 | | | |
| 18 | 6.7 | 476 | 490 | 6.61 |
| | 6.6 | | | |
| | 5.4 | | | |
| 19 | 6.6 | 490 | 504 | 6.23 |
| | 5.3 | | | |
| | 5.6 | | | |
| 20 | 6.8 | 504 | 504 | 3.70 |
| | 6.2 | | | |
| | 6.3 | | | |
| 21 | 6.4 | 504 | 518 | 4.73 |
| | 6.4 | | | |
| | 6.8 | | | |
| 22 | 6.1 | 518 | 532 | 6.67 |
| | 6.4 | | | |
| | 5.1 | | | |
| 23 | 6.2 | 532 | 532 | 5.12 |
| | 4.8 | | | |
| | 6.2 | | | |
| 24 | 6.6 | 532 | 532 | 3.69 |

The first 4 columns are a numerical example with a conventional titration algorithm-here a 14-0-14 algorithm. Column 2 shows the SMBG for the actual day in the week as well as the SMBG for the previous 2 weeks, the titration value being calculated as the average of the 3 measurements. The Bayesian approach is used as an extra level of protection to predict the probability of hypoglycaemia for the 14-0-14-based dose. Correspondingly, the Bayesian approach can be used to ‘reject’ an increase in dose due to a large increase in the probability of hypoglycaemia. As appears, there is a tendency that if an increase in dose decreases the SMBG then the probability of hypo increases.

When comparing tables 1 and 2 it appears that the predicted probabilities of hypoglycaemia are much higher in table 2 than in table 1. This is partly based on the algorithm behind table 1 being optimized to minimize these values (and also minimize the probability of hyperglycaemia) whereas the algorithm behind table 2 is optimized for neither. The probabilities also depend on the particular hyperparameters chosen to describe the uncertainty band for the individual algorithm and also depends on the particular kernel chosen. These parameters are not identical for the two algorithms, in part because the table 1 algorithm is designed for use with a once-daily insulin, e.g. insulin degludec, whereas the table 2 algorithm is designed for use with a once-weekly insulin, e.g. insulin icodec.

1. A computing system for providing a titration dose guidance recommendation for a query subject to treat diabetes mellitus, wherein the system comprises one or more processors and a memory, the memory comprising:
A) instructions for executing by the one or more processors:
a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject,
a probability algorithm adapted to, based on predicted mean and variance FBG values and a pre-defined FBG target range, calculate as a function of insulin dose size the probability of hypoglycaemia respectively hyperglycemia for the subject, and
a policy algorithm adapted to, based on calculated hypoglycaemia respectively hyperglycemia probabilities, calculate the probability for a corresponding policy target as a function of insulin dose size,
B) instructions that, when executed by the one or more processors, perform a method responsive to receiving a dose guidance request (DGR), the method comprising:
obtaining for the subject an FBG target to be used as the pre-defined FBG target range for the probability algorithm,
obtaining from the subject an update data set comprising a most recent FBG value and a corresponding insulin dose size,
using the prediction algorithm: calculating for the subject, based on the update data sat, the mean and the variance of the resulting FBG for the subject as a function of the size of an injected dose of insulin,
using the probability algorithm: calculating as a function of insulin dose size the probability of hypoglycaemia respectively hyperglycemia for the subject,
using the policy algorithm: calculating the probability for the policy target as a function of insulin dose size, and
determining the insulin dose size having the highest calculated probability for meeting the policy target, the determined dose size representing the requested dose size recommendation.

2. A computing system as in claim 1, wherein the memory comprises a historic data set for the subject.

3. A computing system as in claim 1, comprising a smartphone with a display, the display being controlled to display a determined dose size representing the requested dose size recommendation.

4. A computing system for providing a titration dose guidance recommendation for a query subject to treat diabetes mellitus, wherein the system comprises one or more processors and a memory, the memory comprising:
A) instructions for executing by the one or more processors:
a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject,
a policy algorithm adapted to, based on predicted mean and variance FBG values, calculate the probability for a corresponding policy target as a function of insulin dose size,
B) instructions that, when executed by the one or more processors, perform a method responsive to receiving a dose guidance request (DGR), the method comprising:
obtaining for the subject an FBG target to be used as the policy target for the probability algorithm,
obtaining from the subject an update data set comprising a most recent FBG value and a corresponding insulin dose size,
using the prediction algorithm: calculating for the subject, based on the update data sat, the mean and the variance of the resulting FBG for the subject as a function of the size of an injected dose of insulin,
using the policy algorithm: calculating the probability for the policy target as a function of insulin dose size, and
determining the insulin dose size having the highest calculated probability for meeting the policy target, the determined dose size representing the requested dose size recommendation.

5. A method for providing a titration dose guidance recommendation for a query subject to treat diabetes mellitus, the method comprising the steps of:
obtaining for the subject an FBG target to be used as a pre-defined FBG target range for a probability algorithm,
obtaining from the subject an update data set comprising a most recent FBG value and a corresponding insulin dose size,
using a for the subject representative adaptive prediction algorithm:
calculating for the subject, based on the update data set, the mean and the variance of the resulting FBG for the subject as a function of the size of an injected dose of insulin,
using a probability algorithm: calculating as a function of insulin dose size the probability of hypoglycaemia respectively hyperglycemia for the subject based on the predicted mean and variance FBG values and the pre-defined FBG target range,
using a policy algorithm: calculating, based on the calculated hypoglycaemia respectively hyperglycemia probabilities, the probability for a corresponding policy target as a function of insulin dose size, and
determining the insulin dose size having the highest calculated probability for meeting the policy target, the determined dose size representing a dose size recommendation.

6. A method as in claim 5, wherein the update data set is obtained using a historic data set for the subject and a most recent FBG value and a corresponding insulin dose size for the subject.

7. A computing system for providing a titration dose guidance recommendation for a query subject to treat diabetes mellitus, wherein the system comprises one or more processors and a memory, the memory comprising:
A) instructions for executing by the one or more processors:
a for the subject representative prediction algorithm adapted to calculate the mean and the variance of the resulting fasting blood glucose value (FBG) for the subject as a function of the size of an injected dose of insulin, the prediction algorithm being adaptive in response to obtained FBG and corresponding insulin dose size data for the subject,
a probability algorithm adapted to, based on predicted mean and variance FBG values and a hypoglycaemia target value, calculate as a function of insulin dose size the probability of hypoglycaemia for the subject,
B) instructions that, when executed by the one or more processors, perform a method responsive to receiving a dose guidance request (DGR), the method comprising:
obtaining from the subject an update data set comprising a most recent FBG value and a suggested insulin dose size,
obtaining for the subject a threshold acceptable probability for hypoglycaemia,
using the prediction algorithm: calculating for the subject, based on the update data set, the mean and the variance of the resulting FBG for the subject as a function of the size of an injected dose of insulin,
using the probability algorithm: calculating for the suggested insulin dose size the probability of hypoglycaemia for the subject,
determining whether the suggested insulin dose size is above or below the threshold acceptable probability, and
communicating to the subject whether the suggested insulin dose size is acceptable or should be lowered.
